(19) United States
(12) Patent Application Publication
(10) Pub. No.: US 2006/0183680 A1
(43) Pub. Date: Aug. 17, 2006

(54) Remedy for ischemia

(76) Inventors: Yasuhiko Tabata, Kyoto (JP); Masashi Komeda, Kyoto (JP)

Correspondence Address: OSHA LIANG L.L.P., 1221 MCKINNEY STREET, SUITE 2800, HOUSTON, TX, 77010, US

(21) Appl. No.: 10/551,607

(22) PCT Filed: Apr. 1, 2003
(86) PCT No.: PCT/JP03/04165

§ 371 Date: Sep. 30, 2005

(51) Int. Cl. A61K 38/38

(52) U.S. Cl. 514/12

(57) ABSTRACT

The present invention provides a ischemia therapeutic agent that contains vascularization induction factor and gelatin hydrogel, and gradually releases vascularization induction factor, which is useful in treating ishchemia accompanying peripheral circulatory disorders etc. encountering as complications of arteriosclerosis obliterans, Buerger's disease, diabetes and collagen disease.

### TECHNICAL FIELD

The present invention relates to a ischemia therapeutic agent that contains a vascularization induction factor and a gelatin hydrogel, and gradually releases vascularization induction factor.

### BACKGROUND ART

In the field of vascular surgery, numerous cases have been reported involving serious ischemia of the upper or lower extremities caused by peripheral vasculitis associated with atherosclerosis obliterans, thromboangitis obliterans (Buerger's disease) and collagen disease.

Although serious upper or lower extremity ischemic diseases as such are not lethal, the resulting amputation of an upper or lower extremity causes immense psychological and physical suffering for the patient.

Moreover, serious ischemia of the extremities not only causes poor circulation, but also causes delayed wound healing and refractory infections at the affected area, and for example, can cause infection of artificial blood vessels in patients who have undergone bypass surgery using artificial blood vessels, which can ultimately lead to a fatal outcome.

However, there are currently no known treatment methods for this condition that have demonstrated adequate therapeutic effects.

A known example of a surgical treatment method is lower extremity vascular reconstruction. In addition, the application range of this treatment method is tending to expand to also include elderly patients and patients complicated with diseases involving other organs, for which it was difficult to apply this treatment in the past. However, a large number of unsuccessful cases of have been reported due to increases in the patient population resulting from improved diagnostic techniques, and there have even been cases reported which have ultimately resulted to upper or lower extremity amputation. Namely, in serious cases, the therapeutic effects of surgical treatment methods are only able to somewhat extend the period during which the extremities are able to be conserved, and their treatment results have been extremely poor.

On the other hand, although medicinal treatment methods primarily consist of promotion of collateral circulation through administration of circulation ameliorants such as vasodilators, there is currently no known treatment method for which adequate effects can be obtained.

Treatment methods for ischemic diseases are recently being developed that utilize vascularization induction factors.

Vascularization induction factors (vascularization promotion factors) are observed in mature individuals during the course of the progression of a disease state such as in wound healing, growth and metastasis of solid cancers, chronic inflammations and retinopathy. These factors promote the destruction of the basal membranes of venules following existing capillaries, germination of endothelial cells from areas of local destruction, migration and proliferation outside vessels, and various processes of lumen formation, and have activity that promotes the formation of new capillaries and small blood vessels. Typical examples of these factors include basic fibroblast growth factor (bFGF), vascular endothelial growth factor (VEGF), hepatocyte growth factor (HGF), angiotensin, platelet-derived growth factor (PDGF) and ephrin. bFGF is used in the US and Europe a the clinical treatment level for the treatment of ischemic heart disease. In addition, bFGF is also used in Japan at the clinical application level in the field of dermatology, and at the clinical trial level in the field of orthopedic surgery.

A treatment method for ischemic diseases has also been developed that uses genes such as VEGF and HGF. This treatment method consists of administering the gene primarily into muscle, causing the gene to be incorporated in muscle cells, and causing the expression product of the inserted gene in the form of protein to be secreted from the cells containing the gene. This method is characterized by gradual release using cells, or in other words, causing cells to gradually release a vascularization induction factor. However, this method has the shortcomings of the gene expression efficiency being low, and being unable to control the level or timing of gene expression. In addition, there is also the problem of the expression of unknown effects resulting from gene insertion still not having been resolved.

In other words, the most important factor in terms of solving the aforementioned problems is the gradual release of vascularization induction factor. The reason for attempting to secrete a cell growth factor from cells using a gene and obtain the effects of its gradual release is that, in the case of administering a vascularization induction factor in the form of an aqueous solution, expression of the action of the vascularization induction factor is not observed at all, and the vascularization induction factor itself is unable to be gradually released.

If it were possible to gradually release a cell growth factor as in the present invention however, it would be meaningless to select a method that uses a gene, and the aforementioned problems would be able to be solved.

The inventors of the present invention surprisingly found that a preparation that contains a vascularization induction factor and a gelatin hydrogel and gradually releases vascularization induction factor is useful for the treatment of ischemia of the upper or lower extremities, and that a treatment method using this preparation is less invasive and more potently increases blood flow to seriously affected upper and lower extremities by promoting collateral circulation as compared with known treatment methods of the prior art, thereby leading to completion of the present invention.

### DISCLOSURE OF THE INVENTION

Thus, the object of the present invention is to provide an ischemia therapeutic agent that contains vascularization induction factor and gelatin hydrogel, and gradually releases vascularization induction factor.

A gelatin used in the present invention differs from commercially available gelatin and is a gelatin that has the following physical properties:

(1) an acidic gelatin obtained from collagen by alkaline hydrolysis treatment;

(2) molecular weight under non-reducing conditions of SDS-PAGE of about 100,000 to about 200,000 daltons; and,

(3) the zeta potential in aqueous solution of about −15 to about −20 mV.

Although examples of commercially available gelatins include the type A gelatin manufactured by Sigma and the gelatin manufactured by Wako Pure Chemical Industries, the zeta potential in aqueous solution differs in the manner shown below.

Sigma Type A Gelatin: Roughly 0 to roughly 5 mV

Wako Gelatin: Roughly −5 to roughly −2 mV

The zeta potential is an indicator that represents the degree of electrostatic charge of a substance (gelatin), and is considered to be suitable as an indicator of a gelatin that forms an electrostatic complex with HGF in the present invention.

A gelatin of the present invention is obtained by alkaline hydrolysis from a part such as the skin or tendon of various animal species such as cows, from collagen, or from a substance used as collagen. Preferably, it is an acidic gelatin prepared by alkaline treatment of type I collagen originating in bovine bone, and can also be acquired having a sample isoelectric point (IEP) of 5.0 from Nitta Gelatin. Furthermore, although basic gelatin prepared by acid treatment can also be similarly acquired from Nitta Gelatin having an IEP of 9.0, the zeta potential is considerably different as indicated below.
Acidic gelatin (Nitta Gelatin sample IEP 5.0): Roughly −15 to roughly −20 mV
Basic gelatin (Nitta Gelatin sample IEP 9.0): Roughly +12 to roughly +15 mV

A gelatin hydrogel used in the present invention refers to a hydrogel obtained by using the aforementioned gelatin and condensing with various chemical crosslinking agents. Examples of chemical crosslinking agents that can be used include glutaraldehyde, EDC and other water-soluble carbodiimides, propylene oxide, diepoxy compounds and condensation agents. An example of a chemical crosslinking agent that is used preferably is glutaraldehyde.

In addition, the gelatin can also be crosslinked by heat treatment or ultraviolet irradiation.

There are no particular limitations on the form of the gelatin hydrogel, and examples include cylinders, square columns, sheets, disks, spheres and particles. Gelatin hydrogels in the form of cylinders, square columns, sheets and disks are frequently used as implants, while spheres and particles can also be administered by injection.

Gelatin hydrogels in the form of cylinders, square columns, sheets and disks can be prepared by adding a crosslinking agent aqueous solution to a gelatin aqueous solution or adding gelatin to a crosslinking agent aqueous solution, followed by pouring into a mold of a desired shape and allowing the crosslinking reaction to proceed. In addition, a molded gelatin gel may be added directly or after drying to a crosslinking agent aqueous solution. The crosslinking reaction is stopped by contacting with a low molecular weight substance having an amino group such as ethanol amine or glycine, or by adding an aqueous solution having a pH of 2.5 or lower. The resulting gelatin hydrogel is used to prepare a preparation after washing with distilled water, ethanol, 2-propanol or acetone and so forth.

A gelatin hydrogel in the form of spheres or particles can be prepared by, for example, attaching an immobilized stirring motor (for example, the 3-1 motor, EYELA mini D.C. stirrer, manufactured by Shinto Scientific) and Teflon (trade name) propeller to a three-mouth, round-bottom flask, placing the flask and immobilized apparatus in a gelatin solution, adding an oil such as olive oil, stirring at a speed of about 200 to 600 rpm to form a W/O emulsion and adding a crosslinking agent aqueous solution thereto, or dropping the product of pre-emulsifying a gelatin aqueous solution in olive oil (for example, using the Advantec 21 vortex mixer, homogenizer or polytron PT10-35) into olive oil to prepare a fine particulate W/O emulsion, followed by the addition of a crosslinking agent aqueous solution and allowing the crosslinking reaction to proceed. After then recovering the gelatin hydrogel by centrifugal separation, it is washed with ethyl acetate and so forth followed by immersing in 2-propanol or ethanol to stop the crosslinking reaction. The resulting gelatin hydrogen particles are used to prepare a preparation after sequentially washing with 2-propanol, Tween 80-containing distilled water and distilled water.

In the case the gelatin hydrogel particles aggregate, the addition of, for example, a surfactant or ultrasonic treatment (preferably for no more than about 1 minute while cooling) may be carried out.

Furthermore, a fine particulate gelatin hydrogel having a particle size of 20 μm or less can be obtained by pre-emulsification.

The mean particle size of the resulting gelatin hydrogel particles is 1 to 1000 μm, and these particles should be used after sizing to the required size according to the purpose of use.

The following provides an example of another method of preparing a gelatin hydrogel in the form of spheres or particles.

After placing olive oil in the same apparatus as used in the previous example, stirring at a speed of about 200 to 600 rpm, dropping in a gelatin aqueous solution to prepare a W/O emulsion and cooling, ethyl acetate and so forth is added and stirred followed by recovering the gelatin particles by centrifugal separation. After additionally washing the recovered gelatin particles with acetone and ethyl acetate, and then with 2-propanol and ethanol, etc., the particles are allowed to dry. The dry gelatin particles are then suspended in a crosslinking agent aqueous solution containing 0.1% Tween 80, the crosslinking reaction is allowed to proceed while stirring gently, and the particles are washed with 100 mM glycine aqueous solution containing 0.1% Tween 80 or 0.004 N HCl containing 0.1% Tween 80 depending on the crosslinking agent used followed by stopping the crosslinking reaction to prepare gelatin hydrogel particles. The mean particle size of the gelatin hydrogel particles obtained with this method is similar to that in the case of the aforementioned method.

The mechanism of this gradual release is based on vascularization induction factor being physically immobilized on gelatin within the hydrogel. In this state, the factor is not released from the hydrogel. If the gelatin molecules become soluble in water as a result of the hydrogel being decomposed, the immobilized vascularization induction factor is released accompanying that decomposition. Namely, the gradual release properties of the vascularization induction factor can be controlled by the decomposition of the hydrogel. The ease of decomposition of the hydrogel can be changed according to the degree of crosslinking during preparation of the hydrogel.

There are no particular limitations on the conditions of the crosslinking reaction, and it can be carried out, for example, at 0 to 40° C. for 1 to 48 hours.

A gelatin hydrogel of the present invention is such that its moisture content clearly has a considerable effect on the gradual release properties of the vascularization induction factor, and an example of a moisture content that demonstrates preferable gradual release effects is about 80 to 99 w/w %, while a more preferable moisture content is about 95 to 98 w/w %. Moisture content can be used as a measurable indicator of the degree of crosslinking. A large moisture content indicates a low degree of crosslinking as well as greater susceptibility to decomposition. In other words, the value of this moisture content affects the gradual release properties of the vascularization induction factor.

A gelatin hydrogel of the present invention can be used after suitably cutting to an appropriate size and shape, freeze-drying and sterilizing. Freeze-drying can be carried out by, for example, placing the gelatin hydrogel in distilled water, freezing for 30 minutes or more in liquid nitrogen or for 1 hour or more at −80° C., and then drying for 1 to 3 days in a freeze-dryer.

Although the concentrations of gelatin and crosslinking agent when preparing a gelatin hydrogel should be suitably selected according to the desired moisture content, an example of the gelatin concentration is 1 to 20 w/w %, while an example of the crosslinking agent concentration is 0.01 to 1 w/w %.

The vascularization induction factor used in the present invention is a known substance, and that prepared by various methods can be used provided it has been purified to a degree that allows it to be used as a biochemical reagent or pharmaceutical. In addition, a commercially available product (such as Fiblast Spray®) may also be used. An example of a method for producing vascularization induction factor consists of culturing primary cultured cells or established cells that produce vascularization induction factor, separating from the culture supernatant and so forth, and purifying to obtain vascularization induction factor. Alternatively, a gene that encodes vascularization induction factor can be incorporated in a suitable vector using genetic engineering techniques followed by transformation of a suitable host by inserting in said host, and then obtaining the target recombinant induction factor from the culture supernatant of the transformants. There are no particular limitations on the aforementioned host cells, and various host cells conventionally used in genetic engineering techniques can be used, examples of which include E. coli, yeast and animal cells. The induction factor obtained in this manner may have one or multiple amino acids in its amino acid sequence substituted, deleted and/or added, or a sugar chain may be similarly substituted, deleted and/or added, provided it has substantially the same action as naturally-occurring induction factor.

Although any vascularization induction factor can be used for the vascularization induction factor used in the present invention provided it has activity that promotes formation of new capillaries, examples of such vascularization induction factors include basic fibroblast growth factor (bFGF), vascular endothelial growth factor (VEGF), hepatocyte growth factor (HGF), angiotensin, platelet-derived growth factor (PDGF) and ephrin.

A vascularization induction factor gradual release gelatin hydrogel preparation in the present invention refers to a preparation that is obtained by immersing a vascularization induction factor into the aforementioned acidic gelatin hydrogel. Although vascularization induction factor forms a complex with acidic gelatin hydrogel because it is a basic protein, when considering the absorption inhibitory effects of bFGF with respect to the aforementioned changes in ionic strength in solution, this vascularization induction factor gelatin (hydrogel) complex not only involves electrostatic interaction, but is also affected by other interactions such as hydrophobic bonding. The dissociation constant (Kd) of this complex as well as the binding molar ratio of the induction factor to gelatin were obtained according to a Scatchard binding model (Scatchard, G., 1949). The binding molar ratio of bFGF to gelatin is such that roughly 1 bFGF molecule binds to 1 acidic gelatin molecule.

In addition, the Kd value of acidic gelatin at 37° C. is 5.5×10⁻⁷ M, which is about two to three orders larger than the Kd value of heparin sulfate at 20° C. of 1×10⁻⁹ to 2.0×10⁻¹⁰ M (Rahmoune, H. et al., 1988). This indicates that binding of the HGF gelatin complex is weak and not as strong as that between HGF and heparin sulfate.

In the case the molar ratio of a vascularization induction factor such as bFGF, for example, to gelatin is about 1:1 or more, liberation of bFGF occurs easily and the resulting behavior is quite similar to that of free bFGF in terms of activity. However, in the case the molar ratio of bFGF to gelatin is lowered to about 1:1 or less, since the bFGF is adsorbed and the amount that is liberated is reduced, the apparent activity of bFGF appears to decrease.

Thus, although a complex of vascularization induction factor and gelatin or gelatin hydrogel can be made in which the molar ratio between the vascularization induction factor and gelatin is changed in various ways, in order to avoid an initial burst, a preferable example of a complex has a molar ratio in which there are about 1 mole or less of vascularization induction factor to 1 mole of gelatin hydrogel.

Furthermore, the weight ratio of vascularization induction factor to gelatin is preferably about 5 or less, and the weight ratio of vascularization induction factor to gelatin is more preferably about 5 to about 1/10⁴.

Since a vascularization induction factor gradual release gelatin hydrogel preparation of the present invention has vascularization induction factor gradual release effects and stabilizing effects, it is able to demonstrate the function of a vascularization induction factor for a long period of time even in small amounts. Consequently, the inherent function of vascularization induction factors in the form of cardiovascular protective action such as promotion of vascularization, prevention of reperfusion injury and inhibition of fibrosis are able to be demonstrated effectively.

A vascularization induction factor gelatin hydrogel preparation of the present invention can be used parenterally as an injection preparation. It can be administered, for example, subcutaneously, intramuscularly, intravenously, intracelomicly, into connective tissue, intraperiosteally or into a damaged organ.

A vascularization induction factor gradual release gelatin hydrogel preparation of the present invention or complex thereof can be used in a suitable drug form according to the respective application. For example, it can be administered in a drug form such as a sheet, stick, particles, rods or paste. Examples of administration methods include intracutaneous, subcutaneous, intramuscular, intracelomic, into connective tissue and intraperiosteal administration.

Although the dosage of vascularization induction factor in a preparation of the present invention can be suitably adjusted according to the patient's severity, patient's age and body weight, etc., the normal adult dosage is selected from the range of about 0.1 to about 500 μg, and preferably from the range of about 1 to about 100 μg, and can be injected into the affected area or a peripheral site thereof. In addition, said administration can be performed a plurality of times in the case effects are inadequate with a single administration.

A preparation according to the present invention can be applied to treatment of ischemia in the field of vascular surgery. This ischemia is preferably ischemia accompanying a disease selected from the group consisting of atherosclerosis obliterans, Buerger's disease and poor peripheral circulation occurring as a complication of diabetes or collagen disease.

Although a preparation according to the present invention can be applied to the treatment of ischemia caused by poor peripheral circulation, it is preferably applied to the treatment of ischemia in the upper or lower extremities.

### BRIEF DESCRIPTION OF THE DRAWINGS

FIG. 1 shows the normal course of development of collateral circulation in an untreated group designated as group A. Although there are differences between individuals, development of collateral circulation is observed that is the result of intrinsic growth factors even in the absence of treatment. Pre indicates prior to treatment, while post indicates four weeks after treatment.

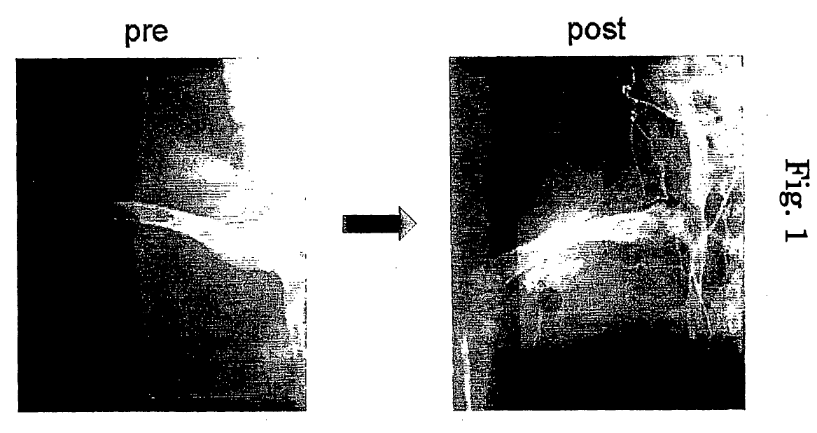

FIG. 2 shows the development of collateral circulation in a group administered gelatin hydrogel only designated as group B. The development of collateral circulation is observed to the same extent as that observed in group A. Pre indicates prior to treatment, while post indicates four weeks after treatment.

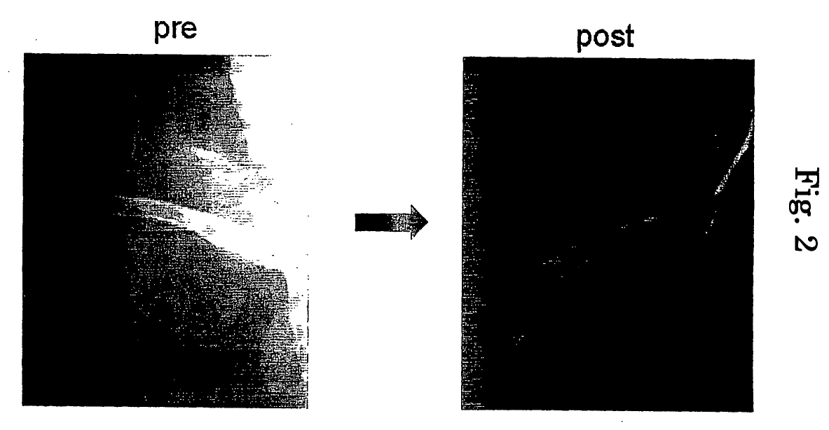

FIG. 3 shows the development of collateral circulation in a group administered gelatin hydrogel containing bFGF (30 μg) designated as group C. The prominent development of collateral circulation paths are observed as a result of administration of bFGF. Pre indicates prior to treatment, while post indicates four weeks after treatment.

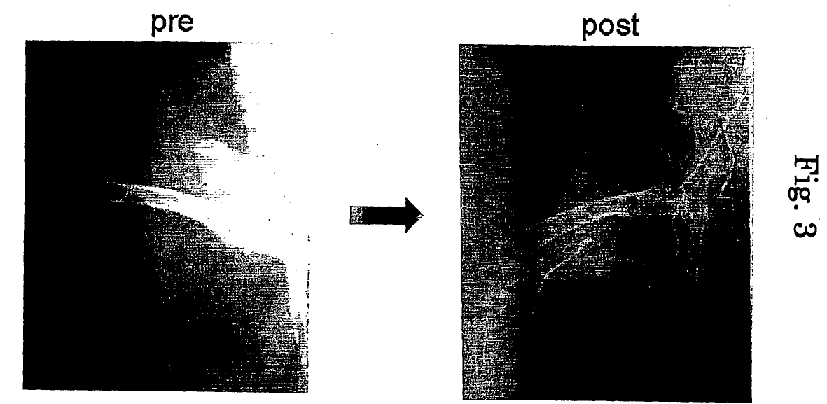

FIG. 4 shows the development of collateral circulation in a group administered gelatin hydrogel containing bFGF (100 μg) designated as group D. Prominent collateral circulation and increased vascularization are observed as a result of administration of bFGF. Pre indicates prior to treatment, while post indicates four weeks after treatment.

FIG. 5 shows tissue specimens of muscle sampled from the femoral region (hematoxylin-eosin staining, magnification factor: 20×). In a comparison between group A and group D, in contrast to extensive capillary formation being observed in group D, capillary formation in group A is observed to be deficient.

FIG. 6 shows the capillary densities of each group (number of capillaries per unit surface area). Significant increases in capillary density are observed that are dependent on the dosage of bFGF (levels of significance: group A vs. D=p<0.0001, group B vs. group D=p<0.0001, group C vs. group D=p<0.0001, group A vs. group C=p<0.05)

### EXAMPLES

A. Preparation of bFGF-Containing Gelatin Hydrogel

Gelatin hydrogel containing bFGF was prepared according to the method described in WO 94/27630. More specifically, a mixture of alkaline-treated gelatin aqueous solution having an isoelectric point of 4.9 (10 wt %, 20 ml) and olive oil (5 ml) was preheated at 40° C. and stirred for 1 hour, and after removing the natural gelatin by cooling the prepared emulsion with ice, acetone was added followed by stirring for 1 hour at 4° C. The resulting gelatin particles were washed three times with acetone (4° C.) and then recovered by centrifugal separation (5000 rpm, 4° C., 5 minutes).

The resulting non-crosslinked gelatin particles (20 mg) were suspended in an aqueous solution of Tween 80 (0.1%, 20 ml) containing glutaraldehyde (0.13 wt %) and then stirred for 24 hours at 4° C. to carry out the crosslinking reaction. After recovering by centrifugal separation (5000 rpm, 4° C., 5 minutes), the particles were stored for 1 hour at 37° C. in glycine aqueous solution (20 ml, 10 mM) and washed three times with distilled water followed by freeze-drying. The mean particle size of the resulting crosslinked gelatin particles was 10 μm. In addition, the moisture content was 95 w/w %.

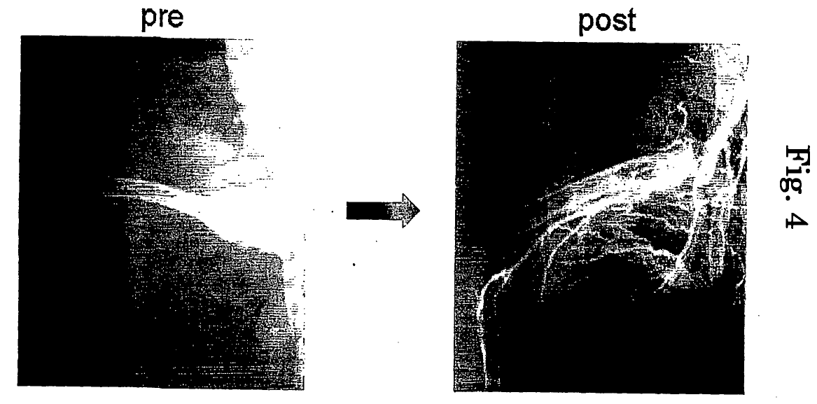

Human bFGF described in FIG. 4 of WO 87/01728 was used for the bFGF, and immersed in the crosslinked gelatin particles by dropping a bFGF aqueous solution (5 mg, 20 μl) into 2 mg of the freeze-dried gelatin particles and allowing to stand for 1 hour at room temperature.

B. Study Using Atherosclerosis Obliterans (ASO) Rabbit Model

1) Preparation of ASO Rabbit Model

The common femoral artery of the right hind leg was ligated on the inside under venous anesthesia and local anesthesia using Japanese white rabbits having body weights of 2.5 to 3.5 kg (males, purchased from Shimizu Laboratory Animals) and then ablated for about 2 cm towards the distal side. The common femoral artery was then also ligated on the distal side and the intermediate portion was completely excised to prepare an ASO model.

Since human ASO is a chronic disease, a two-week progress observation period was established to create a condition that corresponded to this disease.

2) Treatment Method Using bFGF-Containing Gelatin Hydrogel

Angiography was performed on the affected hind legs of all animals in the second week after surgery and used for an evaluation control for comparing the status of lower limb circulation following treatment. The animals were divided into the following four groups following angiography, and a different treatment was performed on each group.

Group A (n=6): Not treated (control group)

Group B (n=6): Administration of gelatin hydrogel only

Group C (n=6): Administration of gelatin hydrogel containing bFGF (30 μg)

Group D (n=6): Administration of gelatin hydrogel containing bFGF (100 μg)

Administration conditions consisted of administration by intramuscular injection into the femoral region of the affected hind leg for the purpose of local exposure to bFGF by gradual exposure for four weeks.

3) Evaluation

Angiography was performed on the affected hind leg following a four-week treatment period, and a sample of muscle was taken from the femoral region of the affected hind leg for histological evaluation.

i) Angiographic Evaluation

The development of collateral circulation was evaluated for each group.

In group A serving as the untreated group, although there were differences between individual animals, development of collateral circulation due to intrinsic growth factors was observed even though these animals were not treated. In group B that was administered only gelatin hydrogel, collateral circulation was observed to have developed to about the same degree as that observed in group A. In group C that was administered gelatin hydrogen gel containing bFGF (30 μg), prominent development of collateral circulation paths was observed as a result of administration of bFGF. In group D that was administered gelatin hydrogel containing bFGF (100 μg), prominent development of collateral circulation as well as increased vascularization were observed as a result of administration of bFGF.

ii) Histological Evaluation

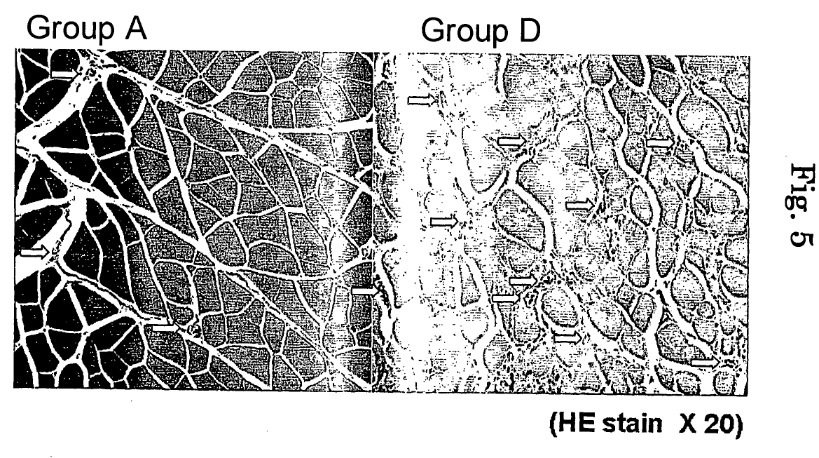

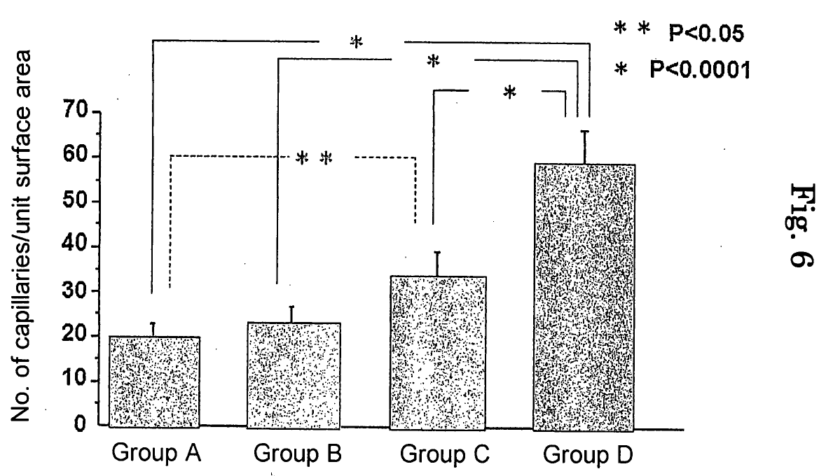

Tissue specimens of muscle sampled from the femoral region (hematoxylin-eosin staining, magnification factor: 20×) are shown in FIG. 5. In contrast to extensive capillary formation being observed in group D, capillary formation in group A was observed to be deficient. Capillary densities between each group (number of capillaries per unit surface area) increased significantly dependent on the dosage of bFGF (FIG. 6).

1. An ischemia therapeutic agent that contains vascularization induction factor and gelatin hydrogen, and gradually releases vascularization growth factor.

2. An ischemia therapeutic agent according to claim 1, wherein the gelatin has the following physical properties:
(1) an acidic gelatin obtained from collagen by alkaline hydrolysis treatment;
(2) molecular weight under non-reducing conditions of SDS-PAGE of about 100,000 to about 200,000 daltons; and,
(3) zeta potential in aqueous solution of about −15 to about −20 mV.

3. An ischemia therapeutic agent according to claim 1, wherein the vascularization induction factor is selected from the group consisting of basic fibroblast growth factor, vascular endothelial growth factor and hepatocyte growth factor.

4. An ischemia therapeutic agent according to claim 1, wherein the ischemia accompanies a disease selected from the group consisting of atherosclerosis obliterans, Buerger's disease and poor peripheral circulation occurring as a complication of diabetes or collagen disease.

5. An ischemia therapeutic agent according to any of claim 1, wherein the ischemia is ischemia in the upper or lower extremities.

6. An ischemia therapeutic agent according to claim 2, wherein the vascularization induction factor is selected from the group consisting of basic fibroblast growth factor, vascular endothelial growth factor and hepatocyte growth factor.

7. An ischemia therapeutic agent according to claim 2, wherein the ischemia accompanies a disease selected from the group consisting of atherosclerosis obliterans, Buerger's disease and poor peripheral circulation occurring as a complication of diabetes or collagen disease.

8. An ischemia therapeutic agent according to claim 3, wherein the ischemia accompanies a disease selected from the group consisting of atherosclerosis obliterans, Buerger's disease and poor peripheral circulation occurring as a complication of diabetes or collagen disease.

9. An ischemia therapeutic agent according to claim 6, wherein the ischemia accompanies a disease selected from the group consisting of atherosclerosis obliterans, Buerger's disease and poor peripheral circulation occurring as a complication of diabetes or collagen disease.

10. An ischemia therapeutic agent according to claim 2, wherein the ischemia is ischemia in the upper or lower extremities.

11. An ischemia therapeutic agent according to claim 3, wherein the ischemia is ischemia in the upper or lower extremities.

12. An ischemia therapeutic agent according to claim 6, wherein the ischemia is ischemia in the upper or lower extremities.

13. An ischemia therapeutic agent according to claim 4, wherein the ischemia is ischemia in the upper or lower extremities.

14. An ischemia therapeutic agent according to claim 7, wherein the ischemia is ischemia in the upper or lower extremities.

15. An ischemia therapeutic agent according to claim 8, wherein the ischemia is ischemia in the upper or lower extremities.

16. An ischemia therapeutic agent according to claim 9, wherein the ischemia is ischemia in the upper or lower extremities.
